(19) United States
(12) Patent Application Publication
(10) Pub. No.: US 2007/0243164 A1
(43) Pub. Date: Oct. 18, 2007

(54) Treating Skin Cancer

(76) Inventor: Amer Kalaaji, Rochester, MN (US)

Correspondence Address: FISH & RICHARDSON P.C., PO BOX 1022, MINNEAPOLIS, MN, 55440-1022, US

(21) Appl. No.: 11/530,307

(22) Filed: Sep. 8, 2006

Related U.S. Application Data
(60) Provisional application No. 60/715,440, filed on Sep. 8, 2005.

(51) Int. Cl. A61K 38/21; A61P 43/00; C07K 14/525; C07K 14/535; C07K 14/56

(52) U.S. Cl. 424/85.7; 530/351

(57) ABSTRACT

This document provides methods and materials related to treating skin cancer. For example, methods and materials relating to the use of cytokines to treat skin cancer are provided.

### CROSS-REFERENCE TO RELATED APPLICATIONS

This application claims the benefit of U.S. Provisional Application Ser. No. 60/715,440, filed Sep. 8, 2005.

### BACKGROUND

1. Technical Field

This document relates to methods and materials involved in treating skin cancer (e.g., melanoma).

2. Background Information

Melanoma is the most serious form of skin cancer. It is a malignant tumor that originates in melanocytes, the cells which produce the pigment melanin that colors skin, hair, and eyes and is heavily concentrated in most moles. It is estimated that 32,100 cases of melanoma were diagnosed in 1995, and that 7,200 patients died of the disease in the United States (Balch et al., Cutaneous melanoma. In: DeVita V J Jr, Hellman S, Rosenberg S A, editors. Cancer: principles and practice of oncology, 5th ed. Philadelphia: Lippincott-Raven; (1997) p. 1947-79). In 2001, there were an estimated 51,400 cases of invasive melanoma and 7,800 deaths due to melanoma (Greenlee et al., CA Cancer J. Clin., 51:15-36 (2001)). In 2005, it is estimated that 59,580 Americans will develop melanoma, and that 7,770 will die of the disease. Thus, melanoma is becoming more common. About 80 percent of patients with stage III melanoma progress to stage IV melanoma, and the median survival rate for patients with stage IV melanoma is seven to nine months (Tomsu et al., Prog. Proc. Am. Soc. Clin. Oncol., 16:495a (1997)). More effective adjuvant therapy is needed for stage III and stage IV melanoma.

### SUMMARY

This document provides methods and materials related to treating skin cancer. For example, the methods and materials provided herein can be used to reduce the progression rate of skin cancer or can be used to induce skin cancer regression. Reducing the progression rate of skin cancer can allow patients to live longer.

This document is based, in part, on the discovery that skin cancer can be treated using a combination of cytokines. For example, a combination of interferon alpha (IFNα), granulocyte-macrophage colony-stimulating factor (GMCSF), and tumor necrosis factor alpha (TNFα) can be used to treat melanoma.

In general, one aspect of this document features a method for treating a mammal having skin cancer. The method comprises, or consists essentially of, administering to the mammal a composition comprising IFNα, GMCSF, and TNFα under conditions wherein the progression rate of the skin cancer is reduced. The mammal can be a human. The skin cancer can be melanoma. The skin cancer can be metastatic melanoma. The composition can be administered by injection. The composition can be administered multiple times. At least one of the IFNα, GMCSF, and TNFα can be a human polypeptide. Each of the IFNα, GMCSF, and TNFα can be a human polypeptide. The progression rate can be reduced by 25 percent. The progression rate can be reduced by 50 percent. The progression rate can be reduced by 75 percent. The progression rate can be reduced by 100 percent.

In another embodiment, this document features a method for treating a mammal having skin cancer. The method comprises, or consists essentially of, administering to the mammal IFNα, GMCSF, and TNFα under conditions wherein the progression rate of the skin cancer is reduced. The mammal can be a human. The skin cancer can be melanoma. The skin cancer can be metastatic melanoma. At least one of IFNα, GMCSF, and TNFα can be administered by injection. IFNα, GMCSF, and TNFα can be administered multiple times. At least one of the IFNα, GMCSF, and TNFα can be a human polypeptide. Each of the IFNα, GMCSF, and TNFα can be a human polypeptide. The progression rate can be reduced by 25 percent. The progression rate can be reduced by 50 percent. The progression rate can be reduced by 75 percent. The progression rate can be reduced by 100 percent.

In another aspect, this document features a composition comprising, or consisting essentially of, IFNα, GMCSF, and TNFα. The composition can contain at least about 100 times more of the GMCSF than the IFNα. The composition can contain at least about 100 times more of the GMCSF than the TNFα. The composition can contain at least about 150 times more of the GMCSF than the IFNα. The composition can contain at least about 150 times more of the GMCSF than the TNFα. The composition can contain between about 100 and 300 times more of the GMCSF than the TNFα and between about 100 and 300 times more of the GMCSF than the IFNα. The composition can contain the same amount of the IFNα and TNFα.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting tumor size versus days post tumor challenge for mice treated with GMCSF (left panel), TNFα (middle panel), or IFNα (right panel).

FIG. 2 is a graph plotting tumor size versus days post tumor challenge for mice treated with a combination of GMCSF, TNFα, and IFNα.

FIG. 3 contains two graphs. The left panel contains a graph plotting the total number of leukocytes in inguinal lymph nodes harvested from both sides of mice that had received intratumoral injections of saline (left side with respect to the mouse) or GMCSF, TNFα, and IFNα (right side with respect to the mouse). The right panel contains a graph plotting the numbers of CD4+ and CD8+ T cells in inguinal lymph nodes harvested from both sides of mice that had received intratumoral injections of saline (left side with respect to the mouse) or GMCSF, TNFα and IFNα (right side with respect to the mouse).

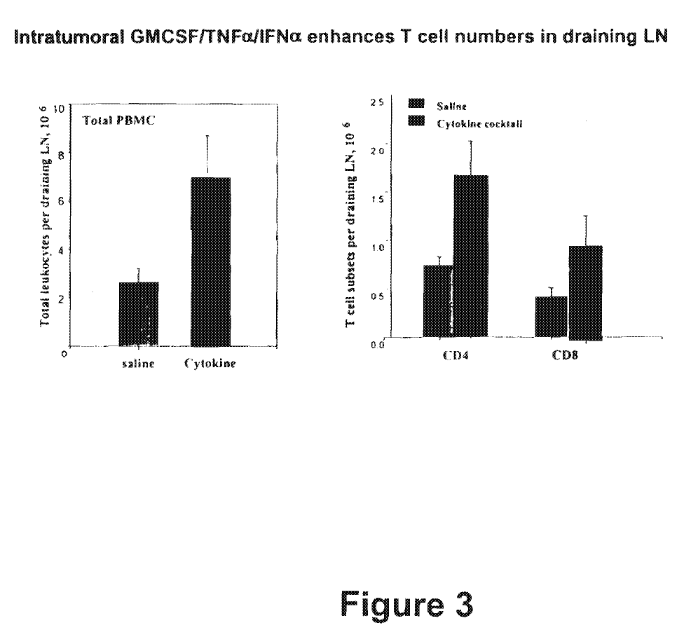

FIG. 4 contains a series of graphs of FACS analyses for cells expressing CD8 and CD107a.

### DETAILED DESCRIPTION

This document provides methods and materials related to treating skin cancer. For example, this document provides methods and materials related to the use of a combination of cytokines to treat skin cancer such as melanoma (e.g., stage I, stage II, stage III, or stage IV melanoma). In some cases, the methods and materials provided herein can be used to treat a single type of skin cancer (e.g., a patient with melanoma) or a combination of skin cancers. A melanoma can be lymph node positive, lymph node negative, or metastatic.

As described herein, a combination of cytokines can be used to treat skin cancer. Such a combination can include, without limitation, IFNα, GMCSF, and TNFα. In general, skin cancer can be treated by administering a composition containing IFNα, GMCSF, and TNFα to a mammal having skin cancer. It will be appreciated that IFNα, GMCSF, and TNFα can be used to treat skin cancer upon administration either individually or in combination. For example, a mammal having skin cancer can be treated by administering IFNα, GMCSF, and TNFα individually.

IFNα can be any polypeptide having IFNα activity, such as human IFNα. For example, IFNα can be recombinant IFNα, synthetic IFNα, isolated IFNα, purified IFNα, commercially available IFNα, or pegylated IFNα. GMCSF can be any polypeptide having GMCSF activity, such as human GMCSF. For example, GMCSF can be recombinant GMCSF, synthetic GMCSF, isolated GMCSF, purified GMCSF, commercially available GMCSF, or pegylated GMCSF. TNFα can be any polypeptide having TNFα activity, such as human TNFα. For example, TNFα can be recombinant TNFα, synthetic TNFα, isolated TNFα, purified TNFα, commercially available TNFα, or pegylated TNFα.

Any type of mammal having skin cancer can be treated using the methods and materials provided herein including, without limitation, mice, rats, dogs, cats, horses, cows, pigs, monkeys, and humans. Any method can be used to administer a composition provided herein to a mammal. For example, a composition provided herein can be administered orally or via injection (e.g., intramuscular injection, intravenous injection, or intracyst injection).

A composition containing a combination of cytokines such as IFNα, GMCSF, and TNFα can be administered following resection of a tumor. In some cases, a composition provided herein can be administered prior to surgical resection of a tumor.

Before administering IFNα, GMCSF, and TNFα or a composition containing a combination of cytokines to a mammal, the mammal can be assessed to determine whether or not the mammal has skin cancer. Any method can be used to determine whether or not a mammal has skin cancer. For example, a mammal (e.g., human) can be identified as having skin cancer using standard diagnostic techniques. In some cases, a tissue biopsy can be collected and analyzed to determine whether or not a mammal has skin cancer.

After identifying a mammal as having skin cancer, the mammal can be treated with IFNα, GMCSF, and TNFα or a composition containing a combination of cytokines such as IFNα, GMCSF, and TNFα. Such compositions can be administered to a mammal in any amount, at any frequency, and for any duration effective to achieve a desired outcome (e.g., to reduce the progression rate of melanoma or to induce cancer regression). In some cases, a composition containing IFNα, GMCSF, and TNFα can be administered to a mammal to reduce the progression rate of melanoma by 5, 10, 25, 50, 75, 100, or more percent. For example, the progression rate can be reduced such that no additional cancer progression is detected. Any method can be used to determine whether or not the progression rate of skin cancer is reduced. For example, the progression rate of skin cancer can be assessed by imaging tissue at different time points and determining the amount of cancer cells present. The amounts of cancer cells determined within tissue at different times can be compared to determine the progression rate. After treatment with a composition provided herein, the progression rate can be determined again over another time interval. In some cases, the stage of skin cancer after treatment can be determined and compared to the stage before treatment to determine whether or not the progression rate was reduced.

An effective amount of a composition provided herein can be any amount that reduces the progression rate of skin cancer without producing significant toxicity to the mammal. Typically, an effective amount can be any amount greater than or equal to about 50 μg provided that that amount does not induce significant toxicity to the mammal upon administration. In some cases, the effective amount can be between 100 and 500 μg. In some cases, a composition can be administered such that the mammal receives between 50 ng and 1 g of IFNα, GMCSF, and TNFα each. In some cases, a composition can be administered such that the mammal receives between 100 and 300 times more of GMCSF than the amount of either IFNα or TNFα. If a particular mammal fails to respond to a particular amount, then the amount can be increased by, for example, ten fold. After receiving this higher concentration, the mammal can be monitored for both responsiveness to the treatment and toxicity symptoms, and adjustments made accordingly. When injected, an effective amount can be between 50 μg and 100 μg. The effective amount can remain constant or can be adjusted as a sliding scale or variable dose depending on the mammal's response to treatment.

Various factors can influence the actual effective amount used for a particular application. For example, the frequency of administration, duration of treatment, use of multiple treatment agents, route of administration, and severity of the skin cancer may require an increase or decrease in the actual effective amount administered.

The frequency of administration can be any frequency that reduces the progression rate of skin cancer without producing significant toxicity to the mammal. For example, the frequency of administration can be from about four times a day to about once every other month, or from about once a day to about once a month, or from about one every other day to about once a week. In addition, the frequency of administration can remain constant or can be variable during the duration of treatment. IFNα, GMCSF, and TNFα and compositions containing such cytokines can be administered daily, twice a day, five days a week, or three days a week. IFNα, GMCSF, and TNFα can be administered for five days, 10 days, three weeks, four weeks, eight weeks, 48 weeks, one year, 18 months, two years, three years, or five years. A course of treatment with IFNα, GMCSF, and TNFα can include rest periods. For example, IFNα, GMCSF, and TNFα can be administered for five days followed by a nine-day rest period, and such a regimen can be repeated multiple times. As with the effective amount, various factors can influence the actual frequency of administration used for a particular application. For example, the effective amount, duration of treatment, use of multiple treatment agents, route of administration, and severity of the skin cancer may require an increase or decrease in administration frequency.

An effective duration for administering a composition provided herein can be any duration that reduces the progression rate of skin cancer without producing significant toxicity to the mammal. Thus, the effective duration can vary from several days to several weeks, months, or years. In general, the effective duration for the treatment of skin cancer can range in duration from several days to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the skin cancer.

A composition containing a combination of cytokines such as IFNα, GMCSF, and TNFα can be in any form. For example, a composition provided herein can be in the form of a solution or powder with or without a diluent to make an injectable suspension. In addition, a composition provided herein can contain a cocktail of cytokines. For example, a composition can contain, without limitation, three, four, five, or more different cytokines. Further, a composition containing a combination of cytokines can contain additional ingredients including, without limitation, pharmaceutically acceptable vehicles. A pharmaceutically acceptable vehicle can be, for example, saline, water, lactic acid, and mannitol.

After administering a composition provided herein to a mammal, the mammal can be monitored to determine whether or not the skin cancer was treated. For example, a mammal can be assessed after treatment to determine whether or not the progression rate of melanoma was reduced (e.g., stopped). As described herein, any method can be used to assess progression rates.

The document will provide addition description in the following examples, which do not limit the scope of the invention described in the claims.

### EXAMPLES

### Example 1

### Materials and Methods

Mice, Cells, and Cytokines: Female C57/BL6 mice that were six to eight weeks of age were obtained from The Jackson Laboratory (Bar Harbor, Me.). The melanoma cell line B16F10 was obtained from American Type Culture Collection (Manassas, Va.). The B16F10 cell line was maintained in Dulbecco's modified Eagle's medium with 10% fetal calf serum, 4 mM L-glutamine, 1.5 g/L sodium bicarbonate, and 4.5 g/L glucose. Recombinant mouse granulocyte-macrophage colony-stimulating factor (GMCSF) was purchased from Cell Sciences (Canton, Mass.). The GMCSF was reconstituted in sterile 20 mM acetic acid, diluted in 0.1% bovine serum albumin (BSA)/phosphate-buffered saline (PBS), aliquotted, and stored at −20° C. Recombinant mouse interferon alpha (IFNα) was purchased from PBL Biomedical Laboratories (Piscataway, N.J.) and stored at −70° C. before being diluted to a working concentration. Recombinant mouse tumor necrosis factor alpha (TNFα) was obtained from Sigma (St. Louis, Mo.). Aliquots of reconstituted TNFα were stored at −20° C.

Assessment of Anti-Tumor Effects: Each hind footpad of five to ten mice per experiment was injected with 1×10⁶ B16F10 cells on day zero. Starting on day three, the right hind footpad of each mouse was injected with 100 μL of a cytokine solution, and the left hind footpad of each mouse was injected with 100 μL of saline, three times per week. Tumor growth was monitored three times per week, and each tumor was measured in two perpendicular dimensions. Tumor size was reported as the product of the two sizes. Various amounts of GMCSF, IFNα, and TNFα were tested individually and in combinations of two or three cytokines. A combination of the three cytokines was tested by mixing 10 μg GMCSF, 50 ng IFNα, and 50 ng TNFα in a final volume of 100 μL per injection. A difference in tumor size between footpads treated with cytokine(s) and saline was evaluated for statistical significance at the 95% confidence interval. Three weeks after tumor challenge, three mice from each treatment group were sacrificed for evaluation of tumor-specific immune responses.

Immunohistochemistry: Both hind footpads of each mouse were removed and immediately frozen in Optimal Cutting Temperature compound (OCT) on dry ice. Tissues sections were cut at 5 μm, fixed in acetone, and stored at −70° C. Anti-mouse CD4 (L3T4) antibodies, anti-mouse CD8a (Ly-2) antibodies, and anti-mouse CD11b antibodies were purchased from BD Pharmingen (San Diego, Calif.). For CD11b staining, the EnVision™ detection system (DAKO Corporation, Carpinteria, Calif.) was used with DAB as the substrate. For CD4 and CD8 staining, the EnVision™ detection system was used with Nova Red (Vector Laboratories, Burlingame, Calif.) as the substrate. Staining was performed according to the manufacturers' recommended protocols.

Evaluation of T Cell Expansion In Vivo: Three weeks after tumor challenge, the draining inguinal lymph nodes were harvested from both the right and the left side of each mouse. The total number of leukocytes was determined by counting the live cells using trypan blue. The percentage of CD4⁺ and CD8⁺ T cells was determined by flow cytometry. The absolute number of CD4⁺ and CD8⁺ T cells per lymph node was calculated by multiplying the percentage of the T cell subset by the total number of leukocytes.

CD107a Mobilization: Harvested lymph node cells were frozen and analyzed in batches. The cells were thawed the day before an experiment and cultured overnight in CTL media (Iscove's modified Dulbecco's medium (IMDM) with 10% fetal bovine serum (FBS), L-glutamine, penicillin/streptomycin, and 100 units/mL IL-2). The following morning, cells were collected, washed, and resuspended in CTL media at a concentration of 1×10⁷ cells/mL for use as effector cells. B16F10 melanoma cells were treated with a trypsin/EDTA solution (Invitrogen Carlsbad, Calif.), washed, and resuspended in CTL media at a concentration of 1×10⁷ cells/mL for use as target cells. Mouse thymoma EL-4 cells were used as control target cells. The ratio of effector to target cells used was generally one to one. All assays were performed in duplicate for each condition using 96-well V-bottom plates. One μL of 2 mM monesin (Sigma) in 100% ethanol was added to each well, followed by addition of 100 μL of target cells (1×10⁶ B16F10 or EL-4 cells), 100 μL of effector cells (1×10⁶ lymph node cells), and 1 μL of FITC-conjugated anti-CD107a (LAMP-1) antibody (BD PharMingen). The contents of each well were mixed using a multichannel pipette. The plate was centrifuged at 1200 rpm for 1 minute and incubated for 6 hours at 37° C. Following the incubation, the plate was centrifuged at 1800 rpm for 1 minute to remove the supernatant. Cell pellets were collected and washed with PBS plus 0.5 mM EDTA and 2% FBS. Cells were stained with PE-conjugated anti-CD8 antibodies (BD PharMingen) and analyzed by fluorescence-activated cell sorting (FACS).

### Example 2

### Anti-Tumor Effects of GMCSF, IFNα, and TNFα

The growth kinetics of tumors derived from B16F10 cells was examined. Mice injected with 1×10⁶ B16F10 cells in each hind footpad were closely monitored. Tumor size in the left footpad was compared to that in the right footpad for 6 weeks. During this time, there were no differences in tumor size or growth curve between the two sides. This established the validity of treating one hind footpad of a mouse with cytokine(s) and using the other hind footpad of the mouse as an internal control. To evaluate the anti-tumor effect of each cytokine individually, GMCSF alone, IFNα alone, or TNFα alone was injected intratumorally into the right hind footpad. As a control, saline was injected intratumorally into the left footpad. Tumor growth was compared between the two sides. Three different amounts of each cytokine were tested. Five μg, 10 μg, and 20 μg of GMCSF were tested. Ten ng, 50 ng, and 100 ng of IFNα were tested. Five ng, 10 ng, and 50 ng of TNFα were tested. The cytokines were injected three times per week for six weeks.

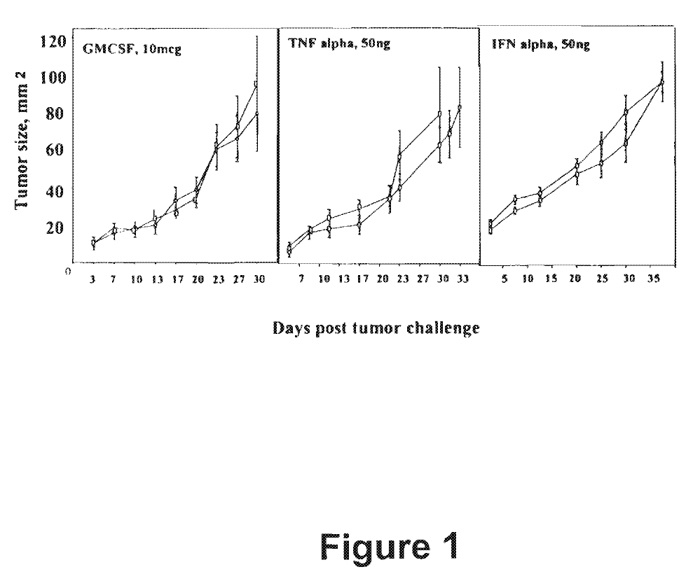

None of the cytokines elicited significant anti-tumor effects when tested individually. There was no difference in tumor size between cytokine-injected and saline-injected footpads in mice treated three times per week for six weeks with 10 μg GMCSF (FIG. 1, left panel), 50 ng TNFα (FIG. 1, middle panel), or 50 ng IFNα (FIG. 1, right panel). Other tested dosages listed above yielded similar results. To determine if there are any synergistic effects between these cytokines, three different combinations of two cytokines were tested: (1) 10 μg GMCSF and 50 ng TNFα, (2) 10 μg GMCSF and 50 ng IFNα, and (3) 50 ng TNFα and 50 ng IFNα. Administration of a combination of two cytokines inhibited tumor growth slightly compared with administration of saline. However, the differences were not statistically significant.

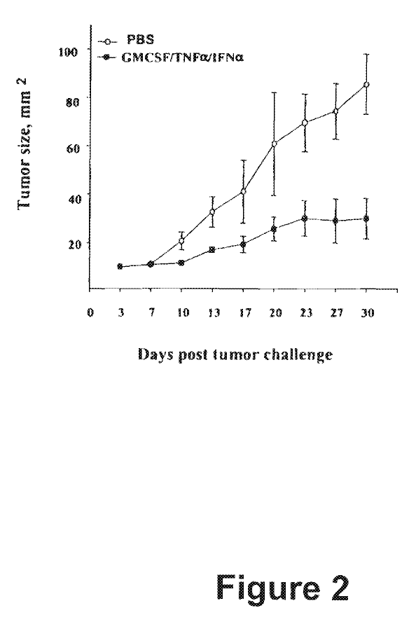

The anti-tumor effect of a combination of three cytokines was also studied. Footpads that were injected intratumorally with a mixture of three cytokines (10 μg GMCSF, 50 ng TNFα, and 50 ng IFNα) had significantly smaller tumors as compared with control footpads (FIG. 2). As presented in FIG. 2, the separation of the tumor growth curve corresponding to the tumor treated with the combination of cytokines from the control tumor growth curve was particularly pronounced after 14 days. The cytokine cocktail-treated tumors grew much slower during the first two to three weeks than the PBS-injected tumors (FIG. 2). In fact, the cytokine cocktail-treated tumors stopped growing after three weeks, in contrast to the uncontrolled growth of the PBS-injected tumors. The experiment was terminated after six weeks because the mice had to be euthanized due to the large ulcerated tumors on their left hind footpads (PBS-injected internal controls). The combination of GMCSF, TNFα, and IFNα could, therefore, induce strong anti-tumor effects that dramatically suppressed tumor growth. Taken together, the results indicate that intralesional injection of the mixture of GMCSF, TNFα, and IFNα greatly potentiated and improved the anti-tumor efficacy of local cytokine therapy. The observed anti-tumor effect of the three-cytokine therapy, but not the two-cytokine therapy, suggested that all three cytokines and their synergistic actions contribute to the overall anti-tumor effect.

### Example 3

### T Cell Expansion in Draining Lymph Node

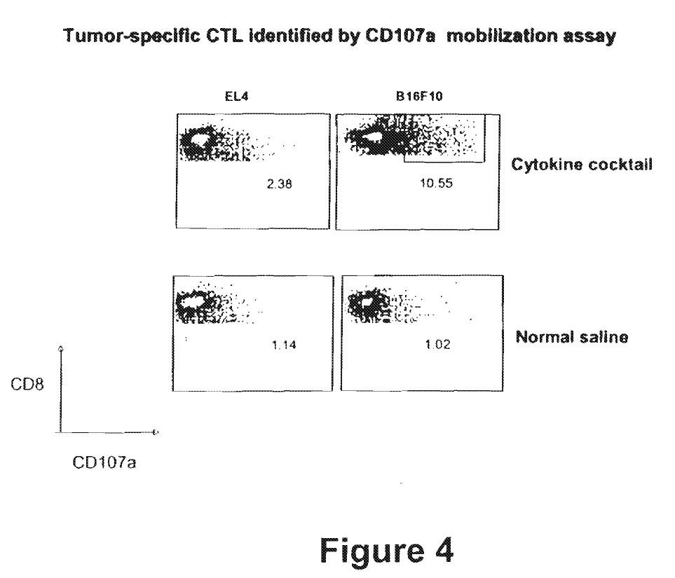

The cytokine cocktail also induced local lymphocytic infiltrate and tumor necrosis. Infiltration of CD8⁺ T cells, CD4⁺ T cells, and CD11b⁺ antigen presenting cells was increased at tumor sites. The significant T cell infiltration at tumor sites suggested that intratumoral injection of the cytokine cocktail could stimulate the proliferation of immune effector cells in situ that contributed to the anti-tumor effects. To determine if any systemic immune response were involved, the secondary lymphoid organs were examined for T cell proliferation. The draining inguinal lymph nodes from both the experimental and control sides were harvested, the leukocytes were counted, and the numbers of CD4⁺ and CD8⁺ T cells were determined. The lymph nodes from the cytokine cocktail-injected side were significantly larger in size than those from the PBS-injected side, and they contained more than twice as many cells (FIG. 4, left panel). After taking into account the percentage of CD4⁺ and CD8⁺ T cell subsets determined by FACS staining, the absolute numbers of both CD4⁺ and CD8⁺ T cells per lymph node increased more than two fold after cytokine cocktail therapy (FIG. 4, right panel). The ratio of CD4 to CD8 cells remained constant in both groups. These results indicated that locally administered cytokine cocktail of GMCSF, TNFα, and IFNα induced a dramatic systemic increase in overall lymphocytes at secondary lymphoid organs including balanced proliferation of both CD4⁺ and CD8⁺ T cells correlated with lymphoadenopathy.

### Example 4

### Generation of Tumor-Specific Cytotoxic T Cell Responses In Vivo

The results above demonstrate that intratumoral administration of GMCSF, TNFα, and IFNα results in T cell proliferation both locally and systemically. To determine if any tumor-specific T cell responses were generated, the T cells from draining lymph nodes were tested for surface mobilization of CD107a upon tumor stimulation using flow cytometry. CD107a (also known as lysosomal-associated membrane-1, LAMP-1) is a vesicle membrane protein that becomes transiently mobilized to the cell surface during the degranulation process when CD8⁺ T cells fuse with a target cell membrane and release cytotoxic mediators, such as perforin and granzymes. Therefore, the CD107a mobilization assay was used to identify tumor-cytolytic T cells based on CD107a mobilization as a marker of degranulation upon interaction with tumor cells without knowing the specific tumor antigen. Cells harvested from inguinal lymph nodes of both sides were first incubated with either B16F10 melanoma cells or control EL4 tumor cells in vitro for 5 hours in the presence of fluorescently-labeled anti-CD107a antibodies. Cells were then stained with anti-CD8 antibodies and analyzed by FACS. The percentage of CD107a⁺/CD8⁺ T cells upon stimulation with B16F10 cells versus control cells was used to measure a tumor-lytic T cell population. As presented in FIG. 5, only CD8⁺ T cells from mice treated with the cytokine cocktail exhibited significant tumor specific cytotoxic function, with 10.55% of CD8⁺ T cells exhibiting degranulation upon recognizing B16F10 cells. After subtracting background killing towards control tumors (2.38%), 8.17% of the cytotoxic T lymphocytes demonstrated recognition of as well as direct killing of melanoma tumor cells within a short period of time. In contrast, T cells from lymph nodes of saline-administered sides had no specific CD107a⁺ staining following incubation with B16F10 cells. The small percentage of CD107a staining, 1.14% in response to B16F10 cells and 1.02% in response to EL4 cells, had no tumor specificity and could be considered background. Both cytokine-treated group and the control group had small percentages of background staining that could be due to the effects of natural killers. The results of the CD107a assay suggest that intratumorally administered GMCSF, TNFα, and IFNα induced systemic tumor-specific T cell responses that played a role in the anti-tumor effects of this therapeutic strategy.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having skin cancer, said method comprising administering to said mammal IFNα, GMCSF, and TNFα or a composition comprising IFNα, GMCSF, and TNFα under conditions wherein the progression rate of said skin cancer is reduced.

2. The method of claim 1, wherein said mammal is a human.

3. The method of claim 1, wherein said skin cancer is melanoma.

4. The method of claim 1, wherein said skin cancer is metastatic melanoma.

5. The method of claim 1, wherein said composition or said IFNα, GMCSF, and TNFα are administered by injection.

6. The method of claim 1, wherein said composition or said IFNα, GMCSF, and TNFα are administered multiple times.

7. The method of claim 1, wherein at least one of said IFNα, GMCSF, and TNFα is a human polypeptide.

8. The method of claim 1, wherein each of said IFNα, GMCSF, and TNFα is a human polypeptide.

9. The method of claim 1, wherein said progression rate is reduced by 25 percent.

10. The method of claim 1, wherein said progression rate is reduced by 50 percent.

11. The method of claim 1, wherein said progression rate is reduced by 75 percent.

12. The method of claim 1, wherein said progression rate is reduced by 100 percent.

13. A composition comprising IFNα, GMCSF, and TNFα.

14. The composition of claim 13, wherein said composition comprises at least about 100 times more of said GMCSF than said IFNα.

15. The composition of claim 13, wherein said composition comprises at least about 100 times more of said GMCSF than said TNFα.

16. The composition of claim 13, wherein said composition comprises at least about 150 times more of said GMCSF than said IFNα.

17. The composition of claim 13, wherein said composition comprises at least about 150 times more of said GMCSF than said TNFα.

18. The composition of claim 13, wherein said composition comprises between about 100 and 300 times more of said GMCSF than said TNFα and between about 100 and 300 times more of said GMCSF than said IFNα.

19. The composition of claim 13, wherein said composition comprises the same amount of said IFNα and TNFα.
